(19) United States
(12) Patent Application Publication
(10) Pub. No.: US 2026/0320797 A1
(43) Pub. Date: Oct. 15, 2026

(54) PROVIDING A CONFIDENCE LEVEL FOR A PROPOSED REGISTRATION OF IMAGES

(71) Applicant: Alcon Inc., Fribourg (CH)

(72) Inventor: Tobias Jura Rapoport, Berlin (DE)

(21) Appl. No.: 19/179,000

(22) Filed: Apr. 15, 2025

(51) Int. Cl. G06T 7/00; A61B 17/00; A61B 34/20; A61B 90/00; A61F 9/007; G06T 7/33; G06T 11/00

(52) U.S. Cl. CPC G06T 7/0014; A61F 9/007; G06T 7/337; G06T 11/00; A61B 2017/00119; A61B 2034/2055; A61B 2034/2065; A61B 2090/364; G06T 2200/24; G06T 2207/20076; G06T 2207/30041; G06T 2207/30168; G06T 2210/41

(57) ABSTRACT

In certain embodiments, a surgical system includes a camera and a computer. The camera captures a site image of a surgical site. The computer receives the site image from the camera, accesses a stored image of the surgical site, and registers the site image and the stored image according to a registration process. The computer then identifies one or more confidence factors of the registration of the images, determines a confidence level of the registration according to the confidence factors, and outputs the confidence level via an output device.

### TECHNICAL FIELD

The present disclosure relates generally to ophthalmic surgical systems, and more particularly to providing a confidence level for a proposed registration of images for ophthalmic surgical systems.

### BACKGROUND

In certain surgical procedures, there may be multiple images of the site of the surgery (e.g., a patient's eye), or the “surgical site”. For example, there may be an image of the surgical site captured during an earlier doctor's visit before the surgery and another image of the surgical site captured just before the surgery begins. As another example, there may be an image of the surgical site that includes markings to assist with the surgery and a live image of the surgical site taken during surgery. The images of the surgical site (e.g., the patient's eye) may be registered, that is, spatially aligned such that points of one image are mapped to corresponding points of the other image.

Different techniques for registering images use different processes and assumptions, so have different rates of successfully registering images. In addition, a variety of factors affect the accuracy of the registration techniques. For example, some techniques register images by matching features or intensity patterns in the images, but the matching process may be negatively affected by poor image quality.

### BRIEF SUMMARY

In certain embodiments, a surgical system includes a camera and a computer. The camera captures a site image of a surgical site. The computer receives the site image from the camera, accesses a stored image of the surgical site, and registers the site image and the stored image according to a registration process. The computer then identifies one or more confidence factors of the registration of the images, determines a confidence level of the registration according to the confidence factors, and outputs the confidence level via an output device.

Embodiments may include none, one, some, or all of the following features:
The confidence factors include a registration metric determined according to a registration feature of the registration process. The registration metric may be determined from a registration process output that indicates the likelihood the registration process properly aligned the images. For example, the registration metric may be determined from a cross-correlation matrix or standard deviation output by the registration process.
The confidence factors include image metric determined according to an image feature of the site image or the stored image. For example, the image metric may be an image quality metric that describes the image quality of the site image or the stored image or may be an image difference metric describing differences between the site image and the stored image.
The computer determines the confidence level by: determining a registration metric determined according to a registration feature of the registration process; determining an image metric determined according to an image feature of the site image or the stored image; and calculating the confidence level according to the registration metric and the image metric.
The computer determines the confidence level by identifying a confidence category of a plurality of confidence categories, each confidence category corresponding to a range of confidence levels. The range of confidence values corresponding to the confidence category may be predetermined. The computer may receive a selection of a range of confidence values corresponding to the confidence category and set the confidence category to include the selected range of confidence values.
The computer outputs the confidence level by displaying a traffic light icon configured to emit a color corresponding to the confidence level.
The computer outputs the confidence level by displaying a number or text representing the confidence level.
The computer outputs the confidence level by emitting speech or a sound representing the confidence level.
After outputting the confidence level, the computer receives input responding to the confidence level. If the input is acceptable, the computer allows a workflow to proceed.
The computer determines if the confidence level satisfies a confidence threshold. If the confidence level does not satisfy the confidence threshold, the computer sends a prompt to the output device to check the registration.
The surgical system includes a surgical device that can perform a surgery at the surgical site. The computer instructs the surgical device to proceed with the surgery in accordance with the confidence level.

In certain embodiments, a surgical system includes a camera and a computer. The camera captures a site image of a surgical site. The computer receives the site image from the camera, accesses a stored image of the surgical site, and registers the site image and the stored image according to a registration process. The computer then identifies confidence factors of the registration of the images. The confidence factors include a registration metric and an image metric. The registration metric is determined according to a registration feature of the registration process, and the image metric is determined according to an image feature of the site image or the stored image. The computer then determines the confidence level of the registration according to the confidence factors and outputs the confidence level via an output device.

Embodiments may include none, one, some, or all of the following features:
The registration metric is determined from a cross-correlation matrix or a standard deviation output by the registration process.
The image metric comprises an image quality metric describing the image quality of the site image or the stored image.
The image metric comprises an image difference metric describing differences between the site image and the stored image.

### BRIEF DESCRIPTION OF THE DRAWINGS

FIG. 1 illustrates an example of a surgical system that can provide a confidence level for image registration, according to certain embodiments;

FIGS. 2A to 2C illustrate examples of the confidence level output to a computer display device of the system of FIG. 1, according to certain embodiments;

FIGS. 3A and 3B illustrate examples of the confidence level output to a speaker of the system of FIG. 1, according to certain embodiments; and

FIG. 4 illustrates an example of a method for providing the confidence level of a proposed image registration as part of a workflow that may be performed by the system of FIG. 1, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Image registration techniques for aligning images have different success rates for properly registering images. Moreover, the success of registering images also depends on factors such as image quality. Accordingly, embodiments disclosed herein provide confidence levels for proposed image registrations. The confidence level for a proposed registration expresses the certainty that the images are correctly or properly registered, e.g., the registration matches human expert opinion. A lower confidence level may indicate that the proposal should be reviewed and possibly adjusted, while a higher confidence level may indicate that the proposal is satisfactory to proceed.

Embodiments of the system may improve the effectiveness and safety of surgical procedures by notifying users of the registration quality, which may increase the likelihood of detecting faulty registrations. The embodiments may also create awareness that registration proposals can be wrong and emphasize the importance of reviewing registrations. The system may also improve efficiency by informing the user how much attention is required for each proposed registration.

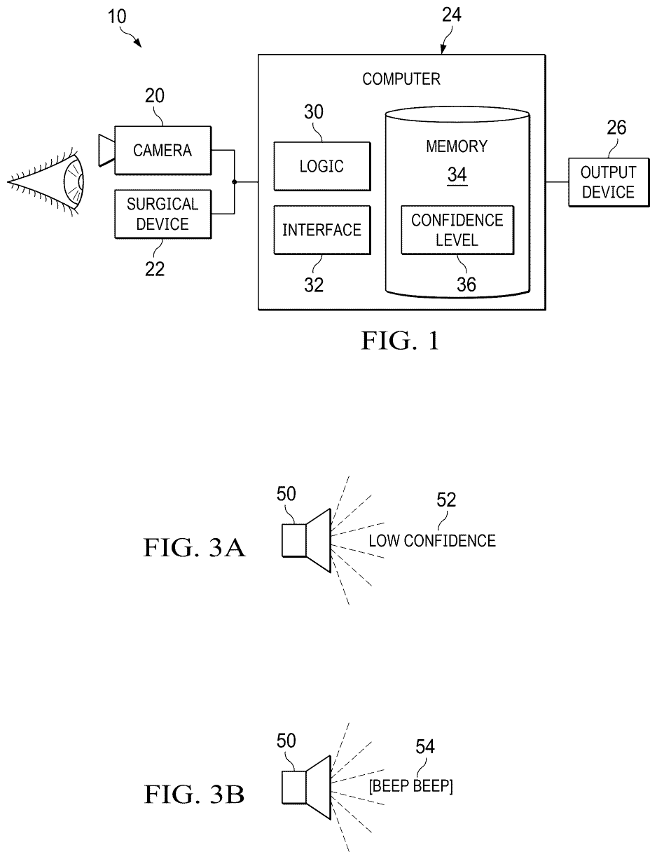

FIG. 1 illustrates an example of a surgical system 10 that can provide a confidence level for image registration, according to certain embodiments. In the example, surgical system 10 includes a camera 20, surgical device 22, computer 24, and output device 26. Computer 24 includes logic 30, an interface 32, and a memory 34 storing applications such as confidence level application 36.

As an overview of the example, camera 20 captures a site image of the surgical site (e.g., a patient eye) of a surgery to be performed using the surgical device 22. Computer 24 receives the site image from camera 20, accesses a stored image of the surgical site, and registers the site image and stored image according to a registration process. Computer 24 then determines the confidence level of the proposed registration of images according to one or more confidence factors and outputs the confidence level to output device 26.

Turning to the components of system 10, in certain embodiments, surgical device 22 is an ophthalmic surgical system used to treat, e.g., an eye. Examples include refractive surgical systems, cataract surgical systems, retinal surgical systems, glaucoma treatment systems, laser surgical systems, and any other suitable surgical system that registers images during treatment. Any suitable images may be registered, such as images of the surgical site, e.g., an eye. The images may be taken at any suitable time, e.g., they may be taken pre-operation (“pre-op”), such as with a diagnostic device prior to surgery, and/or intra-operation (“intra-op”), such as with a surgical device at the start, middle, and/or end of the surgery. For example, an image may be taken pre-op to obtain a diagnostic image, and another image may be taken intra-op at the start of surgery. As another example, an image of an eye may be taken with a diagnostic device, and another image may be taken later with the same device to detect changes in the eye. As yet another example, an image of an eye may be taken with a surgical device at the start of surgery, and another image may be taken later at the end of the surgery to capture changes made by the surgery.

In the embodiments, camera 20 captures a site image of a surgical site (e.g., a patient eye) during a surgical procedure performed with surgical device 22. In general, camera 20 detects light (e.g., visible or infrared light) from (e.g., reflected and/or emitted from) an object and generates a signal in response to the light. The signal carries image data that can be used to generate an image of the object. Examples of camera 20 include charged-coupled device (CCD), video, complementary metal-oxide semiconductor (CMOS) sensor (e.g., active-pixel sensor (APS)), line sensor, and optical coherence tomography (OCT) cameras. In certain embodiments, camera 20 includes one or more cameras.

Computer 24 registers the site image from camera 20 with a stored image of the surgical site. The stored image may be any suitable image of the surgical site, e.g., a diagnostic or other pre-operation image, a treatment image that may include markings that assist with surgery, or a descriptive image that includes a description of the eye or procedure. Computer 24 registers the images according to any suitable registration process, e.g., intensity-based, feature-based, linear or radial transformation, image processing, machine learning, and/or spatial or frequency domain techniques.

Computer 24 uses confidence level application 36 to determine the confidence level of a proposed registration of images. The confidence level indicates the likelihood the registration process has properly aligned corresponding points of the images, e.g., how well the registration output likely matches the optimal registration. The confidence level can be determined according to one or more confidence factors, which may be determined from features of the registration process (“registration features”), features of the site image and/or stored image (“image features”), and/or other suitable features related to image registration. A confidence factor may be expressed as a metric (e.g., a “registration metric” or an “image metric”) that indicates the confidence that images are properly registered.

Registration features may include a registration process output that indicates the likelihood the registration process properly aligned the images. For example, the registration process may use a cross-correlation matrix to register images. A matrix that shows a stronger correlation indicates a higher likelihood of proper registration, which may yield a metric indicating a higher confidence of proper registration. As another example, the registration process may output a standard deviation that describes the variation of the images. A standard deviation that shows less variation indicates a higher likelihood of a proper registration, which may yield a metric indicating a higher confidence of proper registration.

Certain registration features may take into account features of a particular registration process itself that contribute to registration error. For example, a modeling assumption may assume that features of an eye generally match those of the average eye, which might not be the case for, e.g., an extremely damaged eye. As another example, parameters (e.g., optimization or interpolation parameters) may have been generated for one population of eyes (e.g., an adult population), but the parameters may not work for another population (e.g., a newborn population). As another example, a process may select too few features of the eye to properly match different images. A registration metric that takes into account the flaws of a particular registration process may be determined by testing the process (which could utilize machine learning) to determine the process's likelihood of properly registering images.

Image features that affect registration include image quality and image differences. Poor image quality, which may be caused by e.g., noise, distortion, artifacts, or anomalies, may make registering images more difficult. In certain embodiments, computer 24 analyzes distortions, degradations, anomalies, and/or other features of an images to determine an image quality metric. For example, computer 24 may compare a test image (e.g., a site image or a stored image) to a reference image of an eye to determine the quality of the test image. As another example, computer 24 may check that a particular feature of the test image, e.g., the pupil, conforms to the expectation of the feature, e.g., the pupil is circular and black. In the embodiments, poorer image quality yields an image quality metric indicating a lower likelihood of proper registration.

Image differences may make registering images more difficult and can occur in a variety of situations. In one example situation, an eye may have a pathology in one image (e.g., intraoperative image), but not the other (e.g., pre-operative image). In another example situation, images taken under different circumstances (e.g., different types of cameras, different light conditions, or the patient in a different position) may look different (e.g., different brightness, color, or perspective). In certain embodiments, computer 24 compares the images for differences in, e.g., different brightness, color, or perspective, to determine an image difference metric. In the embodiments, a greater image difference yields an image quality metric indicating a lower likelihood of proper registration.

The confidence level can be determined according to one or more confidence factors in any suitable manner. As discussed above, the confidence factors can be expressed as metrics. In certain embodiments, the confidence level L can be determined from a mathematical function of metrics Mᵢ, i=1, . . . , n, where a metric Mᵢ may be a registration, image quality, and/or image difference metric. The function may be, e.g., a weighted sum of the metrics Mᵢ, such as L=Σ¹ₙ ωᵢ Mᵢ, where ωᵢ represents the weight of metric Mᵢ. A metric may have any suitable weight, e.g., a registration metric may have a greater, equal, or lesser weight than the weight of an image metric. In an example, a greater confidence level L indicates greater confidence in a proposed registration. In the example, a registration metric that represents greater confidence in the registration process increases confidence level L, an image quality metric that represents greater image quality increases confidence level L, and an image difference metric that represents greater similarity between images increases confidence level L.

The confidence level may have any suitable form. In certain embodiments, the confidence level may be a number (e.g., a percentage) that expresses the certainty that a registration falls within a confidence interval of any suitable margin of error (e.g., in a range of 1 to 3, 3 to 5, and/or 5 to 10 percent) of being properly registered. In certain embodiments, the confidence level may be a category (e.g., high, medium, low) that represents a range of numbers. Each category represents a different level of certainty and thus may indicate a different level of attention from a user. For example, a high level of confidence category may indicate a quick check of the registration is sufficient; a medium-level category may indicate a more thorough review may be needed; and a low-level category may indicate manual registration may be required. The range of confidence values corresponding to a category may be selected by, e.g., the manufacturer or a user. Computer 24 may receive the selection of the range of values corresponding to a category and set the category to the selected range of values.

In certain embodiments, a confidence threshold may trigger review of proposed registrations. For example, a higher threshold of “check all confidence levels below 100%; accept no levels” presents all registrations for review, and a lower threshold (e.g., “check confidence levels less than 90%; accept levels 90% or greater”) allows proposals to be skipped for review. The confidence threshold may be defined by, e.g., the manufacturer or a user, based on verification and validation of the registration process. In an example, computer 24 determines if a confidence level satisfies a confidence threshold. If the confidence level does not satisfy the confidence threshold, computer 24 sends a prompt to the output device to request that the user check the registration.

In certain embodiments, the process for reporting the confidence level may be part of the workflow of system 10, which may include computer 24 sending instructions to surgical device 22 to perform the surgery. In the embodiments, after outputting the confidence level, computer 24 receives input responding to the confidence level. For example, the user may check and adjust and/or approve the registration. If the input is acceptable, computer 24 allows the workflow to proceed, e.g., computer 24 sends instructions to surgical device 22 to proceed with the surgery. If the confidence level is high (e.g., exceeds a predetermined confidence threshold), the user may be able to skip the review, and computer 24 instructs surgical device 22 to automatically proceed with the surgery. If the confidence level is low, the user may be requested to manually check the proposed registration and provide input indicating the registration has been approved before the workflow can proceed, e.g., before computer 24 instructs surgical device 22 to proceed with the surgery.

In some embodiments, various examples are contemplated for performing or proceeding with the surgery. For example, the instruction may cause a laser to be powered and to convey a laser beam to a target location at a target power and/or repetition rate. As another example, the instruction may cause a phacoemulsification system to receive power. As another example, the instruction may cause a fluid system to provide fluid and/or otherwise maintain pressure in an eye or a portion of the eye. As an additional example, the instruction may cause a cutting tool to begin cutting. In some embodiments, the surgical device 22 may be actively prevented from proceeding (e.g., an electronic governor, an electronic-based safety restraint or switch, etc.) that prevents the surgical device 22 from proceeding to all or a certain stage of the surgery. In these and other embodiments, the instruction may include removing the active prevention such that the surgical procedure may proceed.

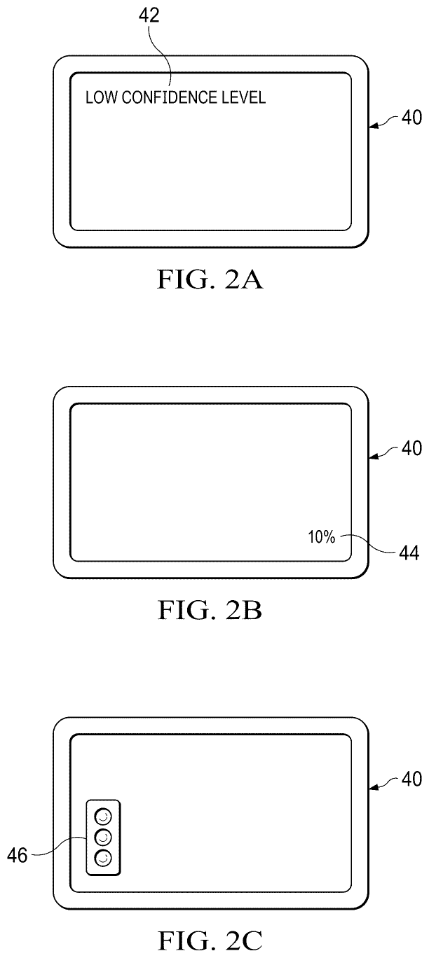

FIGS. 2A to 3B illustrate examples of the confidence level output to an output device 26, according to certain embodiments. Output device 26 may report the confidence level in any suitable format. For example, the output may be visual or auditory. Output device 26 may also provide instructions on how to respond to the confidence level, such as a prompt to check the image registration, e.g., “Please carefully review the registration”.

FIGS. 2A to 2C illustrate examples of the confidence level output to an output device 26 such as a computer display device 40. In certain embodiments, the confidence level may be a number, letter, text, or other symbol that represents a percentage, ranking, or other type of rating. FIG. 2A shows display device 40 providing text 42 indicating a low confidence level. FIG. 2B shows display device 40 providing a number 44 indicating a 10% level. FIG. 2C shows display device 40 providing a traffic light icon 46 that emits a color corresponding to the confidence level. For example, green represents a high level of confidence, yellow a medium level, and red a low level.

FIGS. 3A and 3B illustrate examples of the confidence level output to an output device 26 such as a speaker 50, according to certain embodiments. FIG. 3A shows speaker 50 emitting speech 52 indicating the confidence level. The speech may describe the confidence category, e.g., “Low Confidence”. FIG. 3B shows speaker 50 emitting a sound 54 indicating the confidence level. Sound 54 may be a warning of a confidence level that does not meet a predetermined acceptable threshold, e.g., sound 54 may be a beeping sound.

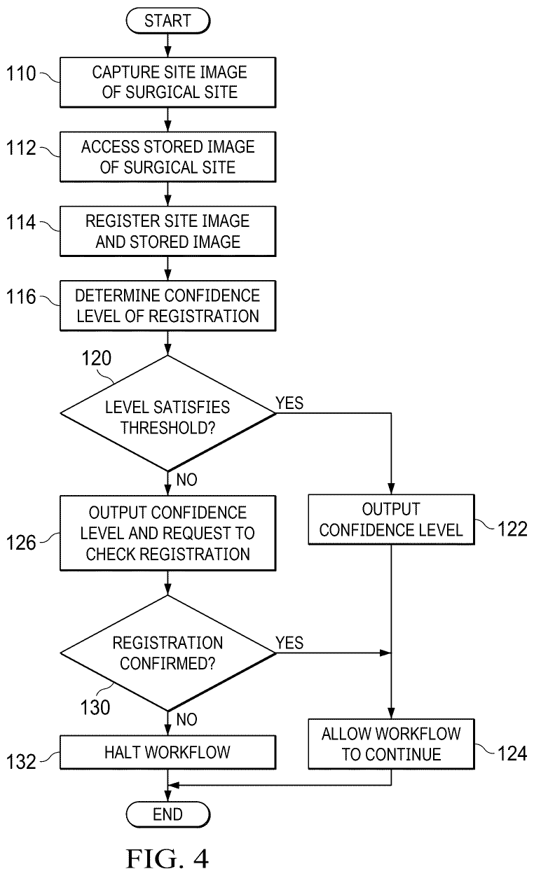

FIG. 4 illustrates an example of a method for providing the confidence level of a proposed image registration as part of a workflow that may be performed by system 10 of FIG. 1, according to certain embodiments. The method starts at step 110, where camera 20 captures a site image of the surgical site. Computer 24 receives the site image from camera 20 and accesses a stored image of the surgical site at step 112. Computer 24 registers the site image and stored image according to a registration process at step 114. At step 116, computer 24 determines the confidence level of the registration according to one or more confidence factors.

The confidence level may satisfy a confidence threshold at step 120. If the level satisfies the threshold, computer 24 outputs the confidence level at step 122 and allows the workflow to continue at step 124. If the level fails to satisfy the threshold, computer 24 outputs the confidence level and requests that the user check the registration at step 126. Computer 24 may receive confirmation that the registration was checked and possibly adjusted at step 130. If computer 24 receives confirmation, it allows the workflow to continue at step 124. If computer 24 fails to receive confirmation at step 130, computer 24 halts the workflow at step 132.

A component (such as the control computer) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display device, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. A surgical system comprising:
a camera configured to capture a site image of a surgical site; and
a computer configured to:
receive the site image from the camera;
access a stored image of the surgical site;
register the site image and the stored image according to a registration process;
identify one or more confidence factors of the registration of the site image and the stored image;
determine a confidence level of the registration of the site image and the stored image according to the one or more confidence factors; and
output the confidence level via an output device.

2. The surgical system of claim 1, wherein the one or more confidence factors comprises a registration metric determined according to a registration feature of the registration process.

3. The surgical system of claim 2, wherein the registration metric is determined from registration process output that indicates a likelihood the registration process properly aligned the site image and the stored image.

4. The surgical system of claim 2, wherein the registration metric is determined from a cross-correlation matrix output by the registration process.

5. The surgical system of claim 2, wherein the registration metric is determined from a standard deviation output by the registration process.

6. The surgical system of claim 1, wherein the one or more confidence factors comprises an image metric determined according to an image feature of the site image or the stored image.

7. The surgical system of claim 6, the image metric comprising an image quality metric describing the image quality of the site image or the stored image.

8. The surgical system of claim 6, the image metric comprising an image difference metric describing one or more differences between the site image and the stored image.

9. The surgical system of claim 1, wherein the computer is configured to determine the confidence level by:
determining a registration metric determined according to a registration feature of the registration process;
determining an image metric determined according to an image feature of the site image or the stored image; and
calculating the confidence level according to the registration metric and the image metric.

10. The surgical system of claim 1, wherein the computer is configured to determine the confidence level by:
identifying a confidence category of a plurality of confidence categories, each confidence category corresponding to a range of confidence levels.

11. The surgical system of claim 10, wherein the range of confidence values corresponding to the confidence category is predetermined.

12. The surgical system of claim 10, wherein the computer is configured to:
receive a selection of range of confidence values corresponding to the confidence category; and
set the confidence category to include the selected range of confidence values.

13. The surgical system of claim 1, wherein the computer is configured to output the confidence level by:
displaying a traffic light icon configured to emit a color corresponding to the confidence level.

14. The surgical system of claim 1, wherein the computer is configured to output the confidence level by:
displaying a number or text representing the confidence level.

15. The surgical system of claim 1, wherein the computer is configured to output the confidence level by:
emitting speech or a sound representing the confidence level.

16. The surgical system of claim 1, wherein the computer is further configured to:
after outputting the confidence level, receive input responding to the confidence level; and
if the input is acceptable, allow a workflow to proceed.

17. The surgical system of claim 1, wherein the computer is further configured to:
determine if the confidence level satisfies a confidence threshold; and
if the confidence level does not satisfy the confidence threshold, send a prompt to the output device to check the registration.

18. The surgical system of claim 1:
further comprising a surgical device configured to perform a surgery at the surgical site; and
wherein the computer is further configured to instruct the surgical device to proceed with the surgery in accordance with the confidence level.

19. A surgical system comprising:
a camera configured to capture a site image of a surgical site; and
a computer configured to:
receive the site image from the camera;
access a stored image of the surgical site;
register the site image and the stored image according to a registration process;
identify a plurality of confidence factors of the registration of the site image and the stored image, the confidence factors comprising a registration metric and an image metric, the registration metric determined according to a registration feature of the registration process, the image metric determined according to an image feature of the site image or the stored image;
determine a confidence level of the registration of the site image and the stored image according to the confidence factors; and
output the confidence level via an output device.

20. The surgical system of claim 19, wherein the registration metric is determined from a cross-correlation matrix or a standard deviation output by the registration process.

21. The surgical system of claim 19, where the image metric comprises an image quality metric describing the image quality of the site image or the stored image.

22. The surgical system of claim 19, where the image metric comprises an image difference metric describing one or more differences between the site image and the stored image.
